(19) United States
(12) Patent Application Publication
(10) Pub. No.: US 2009/0036751 A1
(43) Pub. Date: Feb. 5, 2009

(54) Method and device for acquiring physiological measurement data

(75) Inventors: Theodor Lutze, Balgheim (DE); Dirk Schauer, Berlin (DE)

Correspondence Address: Lipsitz & McAllister, LLC, 755 MAIN STREET, MONROE, CT, 06468, US

(73) Assignee: Aesculap AG, Tuttlingen (DE)

(21) Appl. No.: 12/220,793

(22) Filed: Jul. 28, 2008

Related U.S. Application Data
(63) Continuation of application No. PCT/EP2006/010387, filed on Oct. 28, 2006.

(30) Foreign Application Priority Data
Feb. 1, 2006 (DE) 10 2006 004 523

(51) Int. Cl. A61B 5/00

(52) U.S. Cl. 600/300

(57) ABSTRACT

In a method for acquiring measurement data which are generated by a physiological sensor as a function of physiological parameters of a living being, in which the data generated by the physiological sensor are supplied to an external evaluation unit, which stores these data in a memory, in order to simplify the storing of data when using various sensors, it is proposed that its own memory is associated with each physiological sensor and data originating from the physiological sensor are exclusively stored in the memory associated with the physiological sensor. Moreover, a device for carrying out this method is given.

This application is a continuation of international application number PCT/EP2006/010387 filed on Oct. 28, 2006.

The present disclosure relates to the subject matter disclosed in international application PCT/EP2006/010387 of Oct. 28, 2006 and German application number 10 2006 004 523.8 of Feb. 1, 2006, which are incorporated herein by reference in their entirety and for all purposes.

### BACKGROUND OF THE INVENTION

The invention relates to a method for acquiring measurement data which are generated by a physiological sensor as a function of physiological parameters of a living being, in which the data generated by the physiological sensor are supplied to an external evaluation unit which stores these data in a memory.

Moreover, the invention relates to a device for acquiring measurement data, which are generated by a physiological sensor as a function of physiological parameters of a living being, comprising an evaluation unit, which is connected to the physiological sensor by means of a data transmission path, and comprising a memory device, in which the evaluation unit stores the measurement data supplied by the physiological sensor.

To acquire physiological parameters in the body of a living being, sensors are used, which are either arranged outside the body or are implanted in the body and which, as a function of physiological parameters, generate different electrical signals, which can be received as measurement data by an external evaluation unit. The external evaluation unit is connected, in this case, to the respective physiological sensor by means of a transmission path, which may be a transmission line, but it is also possible for the data transmission to take place wirelessly by means of one of the known transmission methods, in other words, for example, by means of radio signals or by means of optical infrared signals.

The evaluation unit generally stores the measurement data received, so that, on the basis of these measurement data, it is possible to monitor the physiological parameters of the body, optionally even at a later time.

If the external evaluation unit cooperates with various physiological sensors, data which in each case associate the stored measurement data with the physiological sensor from which they originate, have to be input into the evaluation unit, in other words, patient-specific data have to be input into the evaluation unit. This is complicated in the individual case and can lead to errors.

The object of the invention is to develop the above method in such a way that the measurement data received by the various physiological sensors can easily be reliably associated with the respective physiological sensor.

### SUMMARY OF THE INVENTION

This object is achieved according to the invention in a method of the type described at the outset in that each sensor is associated with its own memory and data originating from the physiological sensor is exclusively stored in the memory associated with the physiological sensor.

By providing a separate memory, which is only associated with a single physiological sensor, it is ensured that data originating from this physiological sensor are always only stored in the associated memory without additional patient-related data also having to be input.

According to a particularly advantageous configuration of the invention, it is provided that to check the associated of a memory with a physiological sensor, coding data, which are supplied by the physiological sensor to the evaluation unit, and coding data, which are stored in each memory in each case, are compared with one another and the measurement data supplied by the physiological sensor are only stored in a memory if the coding data of the physiological sensor and of the memory match one another. In this case, an acoustic and/or optic warning signal can be emitted by the evaluation unit.

In this manner, an automatic association of the respective memory with a physiological sensor is obtained on the basis of the coding data which are on the one hand supplied by the sensor to the evaluation unit and, on the other hand, are stored in the associated memory. These coding data may, for example, be an identification number or a letter sequence.

It is favourable if the coding data are stored non-variably in the memory. In this manner, a memory is permanently associated with a certain physiological sensor and this association cannot be cancelled.

In a further preferred embodiment it is provided that calibration data of the associated physiological sensor are stored in the memories, and these are taken into account in the evaluation of the measurement data. The memory thus “knows” certain characteristic variables of the physiological sensor and can take these into account when the measurement data, which are supplied by the physiological sensor and are stored in the associated memory, are evaluated. This may, for example, be the sensitivity of a physiological sensor or a delayed response time or similar parameters. As these data are stored in the associated memory, a confusion of these calibration data cannot occur when receiving measurement data from another physiological memory.

It is particularly advantageous if the various memories are configured as mobile assemblies and, to acquire the measurement data of a physiological sensor, the memory associated with the physiological sensor is connected to the evaluation unit. The evaluation unit, in other words, cannot on its own store the received data of the physiological sensor, but for this purpose needs a special memory which is connected to it. Storage only takes place if this special memory is precisely the memory associated with the physiological sensor, from which data are received.

For example, a memory card or a removable memory, in particular a USB memory module or a compact flash card can be used as the memory.

In a further configuration of the invention it is provided that during the acquisition of measurement data of the physiological sensor, measurement data corresponding to environmental parameters are additionally produced by means of an external sensor, and these measurement data are stored together with the measurement data of the physiological sensor in the memory associated with the physiological sensor. These external parameters may, for example, be environmental pressure or environmental temperature, so during the evaluation, these external parameters can be compared with the measurement data of the physiological sensor.

It may be provided that the data supplied by a physiological sensor to the evaluation unit are only stored in the associated memory if these measurement data satisfy certain plausibility criteria. If the measurement data lie outside a predetermined value range or if the time course of the measurement data differs significantly from a predetermined time course, this may be an indication that the physiological sensor is not working reliably, so storage of the measurement data does not then take place. Instead, a warning signal may be emitted.

It is in particular favourable if comparison data for checking the plausibility criteria are stored in the memories associated with the physiological sensors.

The physiological sensors may determine the most varied physiological parameters particular to the body, in particular it is advantageous if an intracranial pressure sensor which supplies measurement data corresponding to the internal pressure of the brain, is used as the physiological sensor.

The invention is also based on the object of providing a device of the above-mentioned type in such a way that the storage of measurement data supplied by the different physiological sensors by the evaluation unit is simplified.

This object is achieved according to the invention in the device of the type described at the outset in that there is associated with each physiological sensor its own memory, in which the evaluation unit exclusively stores data originating from the physiological sensor.

The evaluation unit is in particular configured in such a way that to check the association of a memory with a physiological sensor, it compares coding data, which are supplied by the physiological sensor to the evaluation unit, and coding data, which are stored in each case in each memory, with one another and only stores the measurement data supplied by the physiological sensor in a memory if the coding data of the physiological sensor and of the memory match one another.

In a preferred embodiment of the invention, it is provided that each memory has a non-variable memory region and a variable memory region, and in that the coding data of the memory are stored in the non-variable memory region.

In a preferred development of the invention it may be provided that calibration data of the associated physiological sensor are stored in the memories and in that the evaluation unit is configured in such a way that it takes into account these calibration data in evaluating the measurement data.

It is particularly advantageous if the various memories are configured as mobile assemblies which can be selectively connected to the evaluation unit and separated again therefrom, in particular, the memories can be configured as flash cards which can be inserted into the evaluation unit.

In a further preferred configuration of the invention it is provided that at least one external sensor which generates measurement data corresponding to environmental parameters, is associated with the evaluation unit, and in that the evaluation unit is configured in such a way that it stores these measurement data together with the measurement data of the physiological sensor in the memory associated with the physiological sensor.

It is furthermore favourable if the evaluation unit is configured in such a way that it only stores the data supplied by the physiological sensor to the evaluation unit in the associated memory if these measurement data satisfy certain plausibility criteria.

In particular, it may be provided here that the evaluation unit is configured in such a way that it stores comparison data for checking the plausibility criteria in the memory associated with the physiological sensors.

The physiological sensor may, for example, be an intracranial pressure sensor, which supplies measurement data corresponding to the internal pressure of the brain.

The following description of preferred embodiments of the invention is used in conjunction with the drawings for a more detailed description.

### BRIEF DESCRIPTION OF THE DRAWINGS

FIG. 1: shows a schematic view of an intracranial pressure sensor and an evaluation unit receiving measurement data of the intracranial pressure sensor;

FIG. 2: shows a simplified view of an evaluation unit;

FIG. 3: shows a block diagram of an evaluation unit;

FIG. 4: shows a graph to show the pressure amplitude of the internal pressure of the brain as a function of the average internal pressure of the brain and

FIG. 5: shows a view of three different time courses of the intracranial pressure amplitude in different brain states.

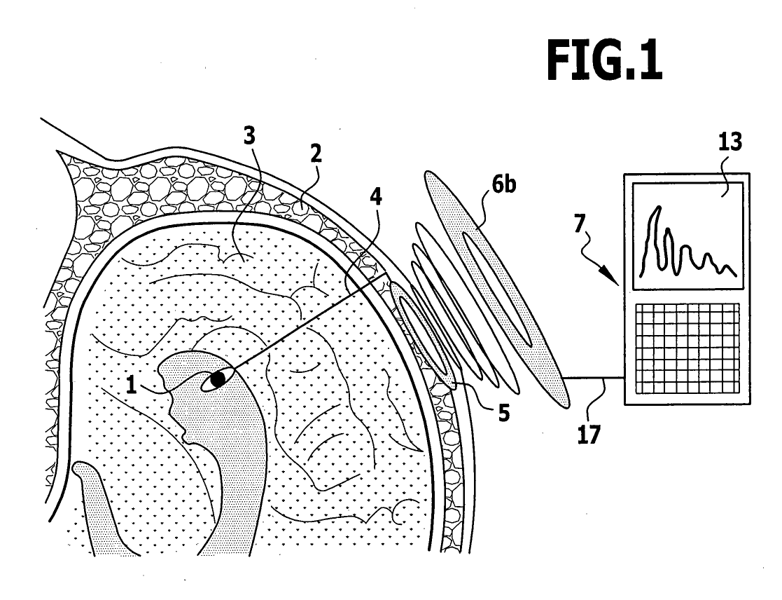

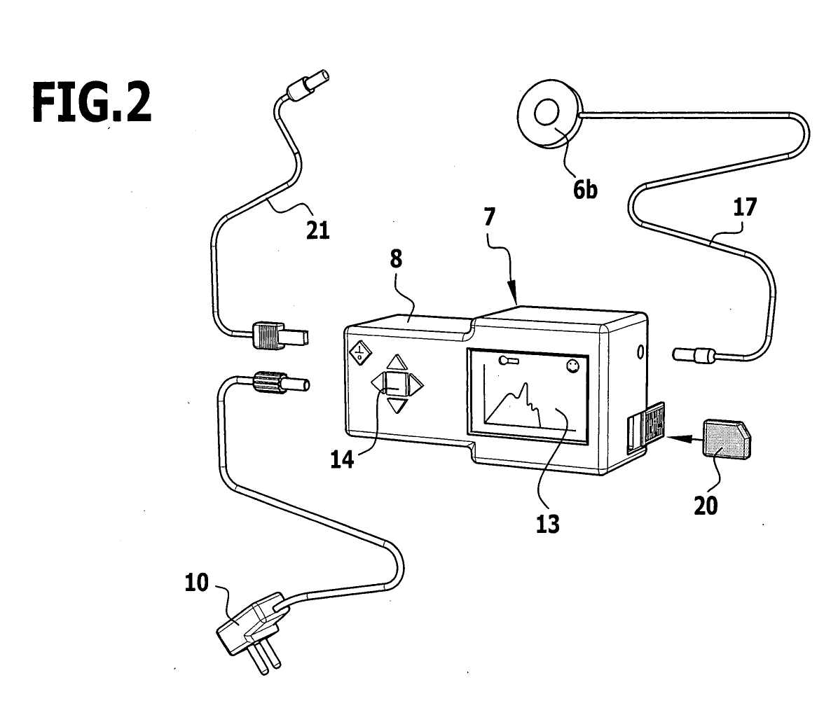

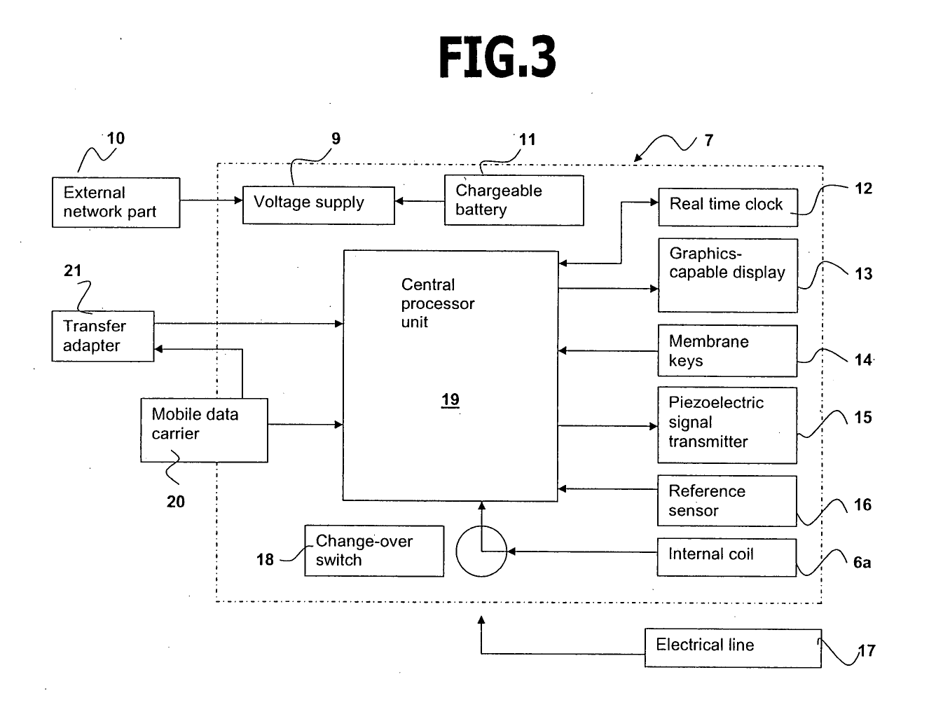

### DETAILED DESCRIPTION OF THE INVENTION

The invention is described using the example of an intracranial pressure sensor 1, which is introduced into the brain 3 through the top of the skull 2 and generates electrical signals depending on the internal pressure of the brain 3, which signals are guided by means of a line 4 to a transmission coil 5 placed on the outside of the top of the skull 2. This transmission coil 5 transmits these measurement data wirelessly to a receiver coil 6a, which is arranged in an evaluation unit 7 or to a receiver coil 6b, which is connected to the evaluation unit 7 by means of a line 17. The evaluation unit 7 is used to store the measurement data received and to display them directly or after evaluation to the treating doctor in such a way that the measurement data supplied by the intracranial pressure sensor 1 provide information about the measurement variables, in other words, in the present example, about the internal pressure of the brain.

The evaluation unit is preferably accommodated as an independent assembly in its own housing 8 and has an electrical voltage supply 9, which is either fed by means of an external network part 10 or by means of a chargeable battery 11. The network part may also be a charging station.

Various units are arranged at the evaluation unit, for example a real time clock 12, a graphics-capable display, for example in the form of a liquid crystal screen, membrane keys 14 for inputting data and control commands, a piezoelectric signal transmitter 15 for generating the necessary carrier frequency for the wireless radio transmission of the measurement data from the transmission coil 5 to the receiver coil 6, and a reference sensor 16, with which external environmental parameters can be determined, for example the environmental pressure.

In the embodiment shown, the measurement data are wirelessly transmitted by the intracranial pressure sensor 1 by means of the line 4 from the transmission coil 5 to the receiver coil 6a or the receiver coil 6b. In another configuration it would be possible to carry out this data transmission with a line connection, by means of a data line, which would then produce a direct connection between the line 4 and the evaluation unit 7. The type of measurement data transmission can be selected by means of a change-over switch 18 arranged in the evaluation unit 7, in other words, a change-over from wireless transmission by means of the receiver coil 6a to conduction by means of the data line 17 is possible as an alternative.

The data line 17 may also, however, be connected to a receiver coil 6b at a distance from the evaluation unit 7, so the change-over switch 18 then changes over between the receiver coil 6a in the evaluation unit 7 and the receiver coil 6b which is connected to the evaluation unit 7 by means of the data line 17.

All the units mentioned are connected to a central processor unit 19 of the evaluation unit 7, in which the data generated by the units and supplied by means of the receiver coil 6 or the data line 17 are processed.

A mobile memory card 20 can be inserted into the evaluation unit 7 by means of a side insertion position and may be configured, for example, as a flash card, known per se. When inserted, this mobile memory card 20 is connected to the central processor unit 19, so the central processor unit can store data on the mobile memory card and if necessary can also read out data.

A central processor unit and optionally also the mobile memory card 20 can optionally be additionally connected by means of a data line 21 to an external data processing apparatus, for example a computer, in which the data received in the evaluation unit and stored in the mobile memory card 20 can be further processed.

The mobile memory card 20 is divided into two memory regions, namely a non-variable memory region, in which data are permanently stored, and in a writable memory region, in which the evaluation unit can store data. The data of the two regions can be read out by the central processor unit.

A coding, by means of which the special mobile memory card 20 can be identified, is permanently stored in the non-variable memory region.

A similar identification coding is also stored in the intracranial pressure sensor 1, and the intracranial pressure sensor 1 together with the measurement data corresponding to the physiological parameters, also sends data, which correspond to this identification coding, to the evaluation unit 7.

The central processor unit compares the codings supplied by the intracranial pressure sensor 1 with the codings of the respectively inserted mobile memory card 20 and only enables this mobile memory card 20 to store measurement data if the codings of the intracranial pressure sensor 1 and the mobile memory card 20 match one another. This match may be an identity, but it is also possible for the conformity to be determined according to various criteria. It is merely essential that only one single memory card 20 is used for one intracranial pressure sensor 1 and has a matching coding. Conversely, for each mobile memory card 20 there is also only a single intracranial pressure sensor 1 with a matching coding, in other words, there is a clear association between the intracranial pressure sensor 1 and mobile memory card 20.

Of course, it is possible for the producer to have a further mobile memory card 20 with an identical coding in store as a replacement, but this may only be used in the event of loss or permanent damage to the current memory card 20, so that it is ensured that the storage of the data originating from a physiological sensor always only takes place on a single memory card 20.

The evaluation unit is therefore only in a position to store the received measurement data of an intracranial pressure sensor 1 if the associated mobile memory card 20 is inserted in the evaluation unit 7 and, if a memory card of this type is missing or another memory card is used, no storage of the measurement data takes place, but, on the other hand, a warning signal can then be generated which alerts the user to the fact that the memory card matching the intracranial pressure sensor 1 has not been inserted.

This ensures that the measurement data originating from an intracranial pressure sensor are always only stored on the same, assigned mobile memory card 20 and that no confusions can occur. Additional inputs with patient data etc. are therefore not necessary.

Further data, for example the time data supplied by the real time clock 12 or measurement data of the reference sensor 16, can be stored on the mobile memory card 20. In this case, the data are also stored on the mobile memory card 20, which corresponds to the intracranial pressure sensor 1 just connected. The instantaneous intracranial pressure values and simultaneously the external environmental pressure values are thus, for example, stored on this memory card, so that the evaluation unit or a connected calculating unit can at any time determine the differential pressure.

Furthermore, calibration data of the intracranial pressure sensor 1 may be stored, preferably in the non-variable memory region, by means of which typical characteristic variables of the intracranial pressure sensor 1 are defined, for example the sensitivity, a temperature dependency or other specific variables which may be significant in the evaluation of the measurement data of the intracranial pressure sensor 1. These characteristic variables specific to the connected intracranial pressure sensor 1 may be taken into account in this manner as they are stored on the assigned mobile memory card 20. Confusions are not possible in this case either and it is moreover not necessary for the user to input specific characteristic variables of the connected intracranial pressure sensor 1 into the evaluation unit 7 by hand. The reliability of operation is thereby considerably increased.

In a preferred development, additional plausibility criteria, which are compared by the central processor unit with the received measurement data, are stored on the respective mobile memory card 20 or else in the central processor unit. If the measurement data received deviate from these plausibility criteria within a predetermined tolerance range, the measurement data are assessed as genuine measurement data and stored in the described manner on the mobile memory card 20 but, on the other hand, if the deviations are greater than expected by the predetermined tolerance range, storage of these measurement data is prevented and a warning signal is emitted. In other words, in a case such as this, the measurement data supplied by the intracranial pressure sensor 1 are not adopted as measurement values, but are merely used to indicate that measurement values are being supplied by the intracranial pressure sensor 1 which do not correspond to the plausibility criteria. This may be caused by a malfunction of the intracranial pressure sensor 1 or else by an abnormal state of the patient. In either case, additional control measures are necessary, which can be carried out by the doctor because of the warning signal.

However, it may also be provided, when the plausibility criteria deviate, that the data supplied by the physiological sensor are stored so that a doctor can check later, in this case, whether the deviation from the plausibility criteria is caused by the apparatus or whether physiological effects are actually responsible for this, which the doctor then in any case has to investigate. It is then preferably provided that the corresponding data are identified on the memory card, so that the doctor, when observing the data, can immediately see that a deviation from the plausibility criteria was present in these data.

The plausibility criteria may, for example, be certain value ranges, within which the measurement values must be located and complementary connections can also be investigated to find suitable plausibility criteria.

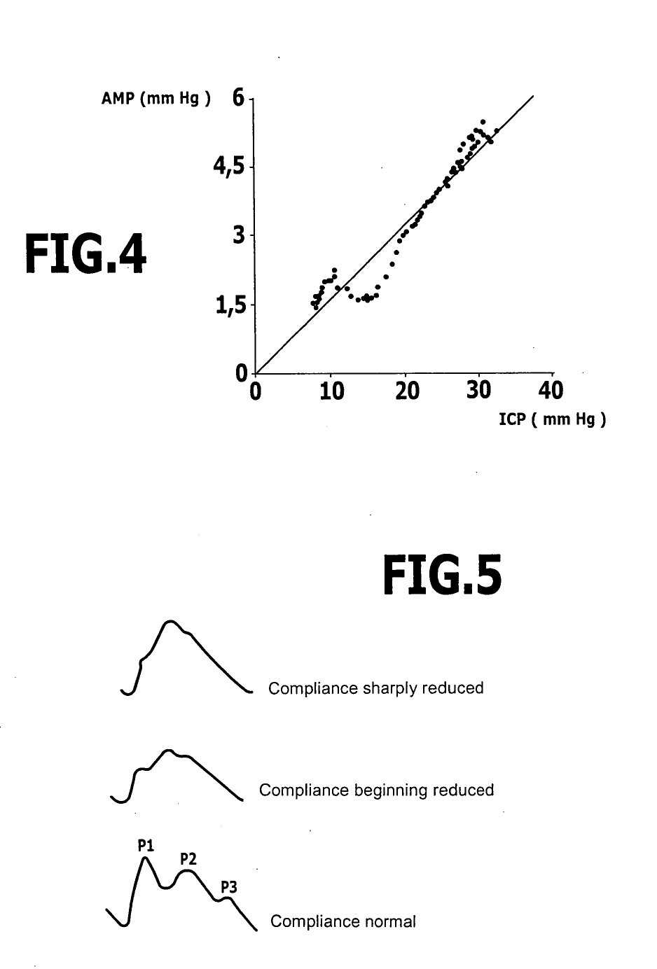

When measuring the intracranial pressure it is possible, for example, to determine a plausibility criterion of this type from a quotient, which compares the amplitude of a pressure pulse in the brain with the average value of the intracranial pressure. This quotient is generally about 0.2 and deviations, which go beyond a predetermined tolerance are a sign that the intracranial pressure sensor 1 is not working reliably or the brain is in an abnormal state. FIG. 4 shows a graph, in which a plurality of measurements of the pressure amplitude and the average pressure are entered in a graph and these measuring points can be approximated by a straight line, which approximately has the desired gradient of 0.2, and this corresponds to the normal state in a functioning intracranial pressure sensor and normally adjusted patient. Greater deviations would be detectable and lead to the said remedial reactions.

A further criterion here is the average value of the intracranial pressure itself which should be between 20 and 60 mm Hg. This can also be checked as a plausibility criterion. A further plausibility criterion can be determined from the time course of the pressure pulse in the brain. The intracranial pressure is determined by arterial inflows and venous outflows, and pulse-synchronous fluctuations of the intracranial pressure are obtained in the process. Normally, the intracranial pressure curve, in other words the time pressure course is characterised by a plurality of individual peaks; a curve of this type is shown with three individual peaks P1, P2 and P3 in FIG. 5 in the lower drawing.

If the state of the brain deviates from the normal state, the individual peaks become increasingly less clear and finally disappear entirely, as is shown in the centre or upper curve in FIG. 5. A comparison of the measured curve shape with curve shapes as shown in FIG. 5, can therefore be used as a plausibility criterion for whether the state of the brain is normal or whether deviations occur which lead to the measured measurement data not being stored on the mobile memory card, but a warning signal being emitted for the doctor.

The use of a mobile memory card, which is associated with a specific intracranial pressure sensor 1, on the one hand, makes it possible to acquire the measurement data from various intracranial pressure sensors 1 with one evaluation unit and to store them exclusively on the associated memory card, but on the other hand it is also possible for a patient with an intracranial pressure sensor 1 to query the corresponding measurement data at various evaluation units, which can then store the corresponding measurement data on the mobile memory card 20 which is carried. In each case, a reliable association is ensured and the operating convenience is improved compared to conventional systems, in which patient data has to be input by hand.

1. Method for acquiring measurement data which are generated by a physiological sensor as a function of physiological parameters of a living being, in which the data generated by the physiological sensor are supplied to an external evaluation unit, which stores these data in a memory, wherein each physiological sensor is associated with its own memory and data originating from the physiological sensor are exclusively stored in the memory associated with the physiological sensor.

2. Method according to claim 1, wherein to check the association of a memory with a physiological sensor, coding data, which are supplied to the evaluation unit by the physiological sensor, and coding data, which are in each case stored in each memory, are compared with one another and the measurement data supplied by the physiological sensor are only stored in a memory if the coding data of the physiological sensor and the memory match one another.

3. Method according to claim 2, wherein the coding data are stored in the memory in a non-variable manner.

4. Method according to claim 2, wherein calibration data of the associated physiological sensor are stored in the memories and these data are taken into account when evaluating the measurement data.

5. Method according to claim 1, wherein the various memories are configured as mobile assemblies and to acquire the measurement data of a physiological sensor, the memory associated with the physiological sensor is connected in each case to the evaluation unit.

6. Method according to claim 5, wherein a flash card which can be inserted in the evaluation unit is used as the memory.

7. Method according to claim 1, wherein when acquiring measurement data of the physiological sensor, measurement data are additionally generated by means of an external sensor, which data correspond to environmental parameters, and these measurement data are stored together with the measurement data of the physiological sensor in the memory associated with the physiological sensor.

8. Method according to claim 1, wherein the measurement data supplied by a physiological sensor to the evaluation unit are only stored in the associated memory if these measurement data satisfy certain plausibility criteria.

9. Method according to claim 8, wherein comparison data for checking the plausibility criteria are stored in the memories associated with the physiological sensors.

10. Method according to claim 1, wherein an intracranial pressure sensor, which supplies measurement data corresponding to the internal pressure of the brain, is used as the physiological sensor.

11. Device for acquiring measurement data, which are generated by a physiological sensor as a function of physiological parameters of a living being, comprising an evaluation unit, which is connected to the physiological sensor by means of a data transmission path, and comprising a memory device, in which the evaluation unit stores the measurement data supplied by the physiological sensor, wherein there is associated with each physiological sensor its own memory, in which the evaluation unit exclusively stores measurement data originating from the physiological sensor.

12. Device according to claim 11, wherein the evaluation unit is configured in such a way that, to check the association of a memory with a physiological sensor, it compares coding data, which are supplied by the physiological sensor to the evaluation unit, and coding data, which are stored in each case in each memory, with one another, and only stores the memory data supplied by the physiological sensor in a memory if the coding data of the physiological sensor and the memory match one another.

13. Device according to claim 12, wherein each memory has a non-variable memory region and a variable memory region and in that the coding data of the memory are stored in the non-variable memory region.

14. Device according to claim 12, wherein calibration data of the associated physiological sensor are stored in the memories and in that the evaluation unit is configured in such a way that it takes into account these calibration data when evaluating the measurement data.

15. Device according to claim 11, wherein the various memories are configured as mobile assemblies which can selectively be connected to the evaluation unit and separated again therefrom.

16. Device according to claim 15, wherein the memory is configured as a flash card which can be inserted in the evaluation unit.

17. Device according to claim 11, wherein at least one external sensor is associated with the evaluation unit and generates measurement data corresponding to the environmental parameters, and in that the evaluation unit is configured in such a way that it stores these measurement data together with the measurement data of the physiological sensor in the memory associated with the physiological sensor.

18. Device according to claim 11, wherein the evaluation unit is configured in such a way that it only stores measurement data, supplied by a physiological sensor to the evaluation unit in the associated memory if these measurement data satisfy certain plausibility criteria.

19. Device according to claim 18, wherein the comparison data for checking the plausibility criteria are stored in the memory associated with the physiological sensors.

20. Device according to claim 11, wherein the physiological sensor is an intracranial pressure sensor, which supplies measurement data corresponding to the internal pressure of the brain.
